(19) United States
(12) Patent Application Publication
(10) Pub. No.: US 2026/0216465 A1
(43) Pub. Date: Jul. 30, 2026

(54) METHOD FOR CONTROLLING OXYGEN GENERATION AND OXYGEN GENERATION SYSTEM

(71) Applicant: BMC (DONGGUAN) MEDICAL CO., LTD., Dongguan (CN)

(72) Inventors: Song WANG, Dongguan (CN); Yantao ZHANG, Dongguan (CN); Wei HE, Dongguan (CN); Zhi ZHUANG, Dongguan (CN)

(21) Appl. No.: 19/038,552

(22) Filed: Jan. 27, 2025

(30) Foreign Application Priority Data
Dec. 6, 2023 (CN) 202311672615.4

(51) Int. Cl. A61M 16/10; A61M 16/00; B01D 53/047

(52) U.S. Cl. CPC A61M 16/101; A61M 16/024; B01D 53/047; A61M 2016/0018; A61M 2016/0027; A61M 2205/702; B01D 2256/12; B01D 2257/102; B01D 2259/4533

(57) ABSTRACT

The present disclosure provides a method for controlling oxygen generation and an oxygen generation system. The method for controlling oxygen generation is applied to the oxygen generation system and includes: detecting an inspiratory event of a user and generating a detection signal corresponding to the inspiratory event; determining an interval duration between a detection time corresponding to a latest detection signal and a current time, and generating a first control signal in response to the interval duration being greater than a first duration threshold; and reducing a latest oxygen generation amount of the oxygen generation system from a first oxygen generation amount to a target oxygen generation amount according to the first control signal, where the first oxygen generation amount is an oxygen generation amount corresponding to a first gear currently set for the oxygen generation system.

### TECHNICAL FIELD

The present disclosure relates to the field of oxygen generation technologies, and more particularly to a method for controlling oxygen generation and an oxygen generation system.

### BACKGROUND

An oxygen generation system can utilize selective adsorption characteristics of molecular sieve and provide power through a compressor to separate nitrogen from oxygen in the air, thereby generating high concentration oxygen. For example, a household or medical oxygenerator, a portable oxygenerator, and the like.

In the related art, the oxygen generation system can provide a certain amount of oxygen to a user according to a set gear when an inspiratory event of the user is detected. If a gear of the oxygen generation system is set relatively high, the amount of oxygen generated is relatively large, a rotating speed of the compressor is relatively high, and power consumption of the oxygen generation system is relatively high.

However, in the related art, the oxygen generation system will continue to operate at the set gear. Even if the inspiratory event of the user is not detected for a period of time, the oxygen generation system will continue to generate oxygen according to an oxygen generation amount corresponding to a current set gear, resulting in excessive power consumption of the oxygen generation system.

### SUMMARY

The present disclosure provides a method for controlling oxygen generation and an oxygen generation system to solve the problem that the existing oxygen generation system continuously generates oxygen according to the oxygen generation amount corresponding to the set gear, resulting in excessive power consumption of the oxygen generation system.

Technical solutions of the present disclosure are as follows.

According to a first aspect of embodiments of the present disclosure, there is provided a method for controlling oxygen generation, applied to an oxygen generation system, including:
detecting an inspiratory event of a user and generating a detection signal corresponding to the inspiratory event;
determining an interval duration between a detection time corresponding to a latest detection signal and a current time, and generating a first control signal in response to the interval duration being greater than a first duration threshold; and
reducing a latest oxygen generation amount of the oxygen generation system from a first oxygen generation amount to a target oxygen generation amount according to the first control signal, where the first oxygen generation amount is an oxygen generation amount corresponding to a first gear currently set for the oxygen generation system.

Optionally, generating the first control signal in response to the interval duration being greater than the first duration threshold includes:
in response to the interval duration being greater than the first duration threshold and less than a second duration threshold, lowering the oxygen generation system from the first gear to a second gear, and generating a first sub-signal corresponding to the second gear; and
reducing the latest oxygen generation amount of the oxygen generation system from the first oxygen generation amount to the target oxygen generation amount according to the first control signal includes:
reducing the latest oxygen generation amount from the first oxygen generation amount to a second oxygen generation amount corresponding to the second gear according to the first sub-signal.

Optionally, generating the first control signal in response to the interval duration being greater than the first duration threshold includes:
generating a second sub-signal in response to the interval duration being greater than the second duration threshold; and
reducing the latest oxygen generation amount of the oxygen generation system from the first oxygen generation amount to the target oxygen generation amount according to the first control signal includes:
reducing the latest oxygen generation amount from the second oxygen generation amount to zero according to the second sub-signal.

Optionally, the method further includes:
generating a second control signal corresponding to the first gear in response to the interval duration being less than a third duration threshold, where the third duration threshold is less than the first duration threshold; and
determining the first oxygen generation amount as the latest oxygen generation amount according to the second control signal, and delivering oxygen to the user according to an oxygen flow rate corresponding to the first gear.

Optionally, determining the first oxygen generation amount as the latest oxygen generation amount according to the second control signal includes:
determining a latest oxygen generation pressure of the oxygen generation system as a target pressure value according to the second control signal, where the target pressure value is an oxygen generation pressure value corresponding to the first oxygen generation amount; and
generating oxygen with the first oxygen generation amount according to the latest oxygen generation pressure.

Optionally, detecting the inspiratory event of the user and generating the detection signal corresponding to the inspiratory event includes:
sampling an inspiratory action of the user according to a preset sampling period; and
in response to a quantity of times of sampled inspiratory actions being greater than a preset quantity-of-times threshold, determining that the inspiratory event of the user is detected, and generating the detection signal corresponding to the inspiratory event, where the preset quantity-of-times threshold is determined according to a breathing frequency of the user.

Optionally, sampling the inspiratory action of the user according to the preset sampling period includes:
detecting a gas pressure value at a target position according to the preset sampling period, and determining a target pressure difference at the target position according to the gas pressure value and an atmospheric pressure value, where the target position is a gas delivery position where the oxygen generation system delivers oxygen to the user; and
determining a first difference value according to the target pressure difference and a basic pressure difference, and determining that one-time inspiratory action of the user is sampled in response to an absolute value of the first difference value being greater than a preset pressure difference threshold, where the basic pressure difference is a pressure difference calibration value at the target position in a preset time interval after the oxygen generation system supplies oxygen to the user.

According to a second aspect of the embodiments of the present disclosure, there is provided an oxygen generation system, including: a detection module, a control module and an oxygen supply module, where the control module is connected to the detection module and the oxygen supply module respectively,
the detection module is configured to detect an inspiratory event of a user, generate a detection signal corresponding to the inspiratory event, and transmit the detection signal to the control module;
the control module is configured to determine an interval duration between a detection time corresponding to a latest detection signal and a current time, generate a first control signal in response to the interval duration being greater than a first duration threshold, and transmit the first control signal to the oxygen supply module; and
the oxygen supply module is configured to reduce a latest oxygen generation amount of the oxygen generation system from a first oxygen generation amount to a target oxygen generation amount according to the first control signal, where the first oxygen generation amount is an oxygen generation amount corresponding to a first gear currently set for the oxygen generation system.

Optionally, the control module is further configured to:
in response to the interval duration being greater than the first duration threshold and less than a second duration threshold, lower the oxygen generation system from the first gear to a second gear, and generate a first sub-signal corresponding to the second gear; and
the oxygen supply module is further configured to reduce the latest oxygen generation amount from the first oxygen generation amount to a second oxygen generation amount corresponding to the second gear according to the first sub-signal.

Optionally, the control module is further configured to:
generate a second sub-signal in response to the interval duration being greater than the second duration threshold; and
the oxygen supply module is further configured to reduce the latest oxygen generation amount from the second oxygen generation amount to zero according to the second sub-signal.

Optionally, the control module is further configured to generate a second control signal corresponding to the first gear in response to the interval duration being less than a third duration threshold, where the third duration threshold is less than the first duration threshold; and
the oxygen supply module is further configured to determine the first oxygen generation amount as the latest oxygen generation amount according to the second control signal, and deliver oxygen to the user according to an oxygen flow rate corresponding to the first gear.

Optionally, the oxygen supply module includes a pressure control unit and an adsorption unit, where the control module is connected to the pressure control unit, and the pressure control unit is further connected to the adsorption unit,
the pressure control unit is configured to determine a latest oxygen generation pressure of the oxygen generation system as a target pressure value according to the second control signal, where the target pressure value is an oxygen generation pressure value corresponding to the first oxygen generation amount; and
the adsorption unit is configured to generate oxygen with the first oxygen generation amount according to the latest oxygen generation pressure.

Optionally, the detection module is configured to:
sample an inspiratory action of the user according to a preset sampling period; and
in response to a quantity of times of sampled inspiratory actions being greater than a preset quantity-of-times threshold, determine that the inspiratory event of the user is detected, and generate the detection signal corresponding to the inspiratory event, where the preset quantity-of-times threshold is determined according to a breathing frequency of the user.

Optionally, the oxygen generation system further includes an air supply pipe, where a first end of the air supply pipe is connected to the oxygen supply module and the detection module respectively, a second end of the air supply pipe is connected to the user, and the detection module is further configured to:
detect a gas pressure value in the air supply pipe according to the preset sampling period, and determine a target pressure difference in the air supply pipe according to the gas pressure value and an atmospheric pressure value; and
determine a first difference value according to the target pressure difference and a basic pressure difference, and determine one-time inspiratory action of the user is sampled in response to an absolute value of the first difference value being greater than a preset pressure difference threshold, where the basic pressure difference is a pressure difference calibration value of the air supply pipe in a preset time interval after the oxygen generation system supplies oxygen to the user.

The technical solutions provided by the embodiments of the present disclosure have at least the following beneficial effects.

In the embodiments of the present disclosure, by detecting the inspiratory event of the user, generating the detection signal corresponding to the inspiratory event and determining the interval duration between the detection time corresponding to the latest detection signal and the current time, an interval duration between the current time and one-time latest inspiratory event of the user can be determined; and then by generating the first control signal in the case where the interval duration is greater than the first duration threshold, and reducing the latest oxygen generation amount of the oxygen generation system from the first oxygen generation amount to the target oxygen generation amount according to the first control signal, the latest oxygen generation amount of the oxygen generation system can be reduced by the first control signal in the case where the interval duration exceeds the first duration threshold and the inspiratory event of the user is not detected. Since the first oxygen generation amount is the oxygen generation amount corresponding to the first gear currently set for the oxygen generation system, by reducing the latest oxygen generation amount from the first oxygen generation amount to a target oxygen generation amount, the problem of excessive power consumption of the oxygen generation system caused by continuously generating oxygen according to the current set gear in the case where the inspiratory event of the user is not detected by the oxygen generation system for a period of time in the related art can be avoid. According to the method for controlling oxygen generation provided in the embodiments of the present disclosure, the power consumption of the oxygen generation system can be reduced by reducing the latest oxygen generation amount of the oxygen generation system.

It should be understood that the above general description and the following detailed description are merely exemplary, and are not restrictive of the present disclosure.

### BRIEF DESCRIPTION OF THE DRAWINGS

The accompanying drawings, which are incorporated in and constitute a part of this specification, illustrate embodiments consistent with the present disclosure and together with the description serve to explain principles of the present disclosure, and do not constitute an undue limitation of the present disclosure.

FIG. 1 is a flowchart of steps of a method for controlling oxygen generation according to an exemplary embodiment.

FIG. 2 is a flowchart of steps of another method for controlling oxygen generation according to an exemplary embodiment.

FIG. 3 is a schematic structural diagram of an oxygen generation system according to an exemplary embodiment.

FIG. 4 a schematic structural diagram of another oxygen generation system according to an exemplary embodiment.

FIG. 5 a schematic structural diagram of a still another oxygen generation system according to an exemplary embodiment.

### REFERENCE NUMERALS

10: oxygen generation system; 101: detection module; 102: control module; 103: oxygen supply module; 104: air supply pipe; 1031: pressure control unit; 1032: adsorption unit; 1033: oxygen output unit.

### DETAILED DESCRIPTION

In order to enable a person skilled in the art to better understand the technical solutions of the present disclosure, the technical solutions in the embodiments of the present disclosure will be clearly and completely described below with reference to the accompanying drawings.

It should be noted that terms such as “first” and “second” in the specification and claims of the present disclosure and the above accompanying drawings are used to distinguish similar objects and do not indicate a particular order or sequence. It should be understood that the terms used in this way can be interchanged in appropriate cases, so that the embodiments of the present disclosure described herein can be implemented in an order other than those illustrated or described herein. The embodiments described in the following exemplary embodiments do not represent all embodiments consistent with the present disclosure. Instead, they are only examples of devices and methods consistent with some aspects of the present disclosure as described in detail in the appended claims.

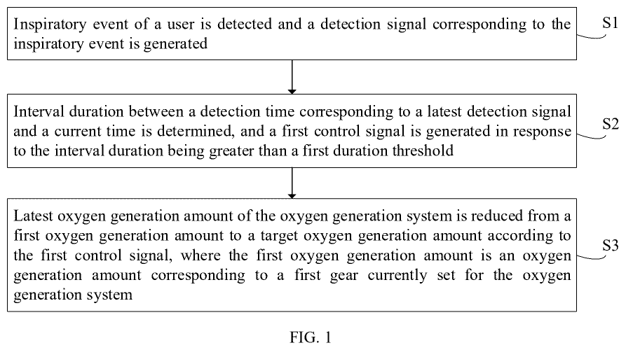

FIG. 1 is a flowchart of steps of a method for controlling oxygen generation according to an exemplary embodiment. As shown in FIG. 1, the method for controlling oxygen generation is applied to an oxygen generation system 10, and the method for controlling oxygen generation can include the following steps.

Step S1, an inspiratory event of a user is detected and a detection signal corresponding to the inspiratory event is generated.

In the embodiment of the present disclosure, the oxygen generation system 10 can be a household or medical oxygenerator, or a portable oxygenerator. The oxygen generation system 10 can adopt pulsed oxygen delivery (POD) technologies, which are characterized by providing oxygen to the user only when an inspiratory event of the user is detected. The oxygen generation system 10 can be connected to an external power supply and be powered by the external power supply, and the oxygen generation system 10 can also include a battery and be powered by the battery. When the oxygen generation system 10 is powered by the battery, if oxygen is continuously generated at a set gear, it will generate relatively excessive power consumption, resulting in unnecessary power consumption of the battery, thereby shortening the effective usage time of the user.

In the embodiment of the present disclosure, the oxygen generation system 10 can detect the inspiratory event of the user by detecting changes of pressure or flow rate in an air supply pipe 104. Specifically, a pressure difference between an inside of the air supply pipe 104 and an external atmospheric pressure can be detected. When it is detected that negative pressure is generated in the air supply pipe 104 and the pressure difference is less than a preset pressure difference threshold, it can be determined that the inspiratory event of the user is detected. Alternatively, a constant and continuous airflow can be delivered in the air supply pipe 104, and the oxygen generation system 10 detects flow rates of airflows at an inlet end and an outlet end of the air supply pipe 104. When a difference between the flow rates of the airflows at both ends reaches a preset flow rate threshold, it is determined that the inspiratory event of the user is detected. This is only an example, and the embodiments of the present disclosure do not limit this.

Optionally, step S1 can include the following steps.

Step S11, an inspiratory action of the user is sampled according to a preset sampling period.

In the embodiment of the present disclosure, the preset sampling period represents a time interval between two samplings, for example, 2 milliseconds (ms). The oxygen generation system 10 samples the inspiratory action of the user at a certain time interval. The inspiratory action of the user can be sampled by referring to the method of detecting the changes of the pressure or the flow rate in the air supply pipe 104 described in the previous embodiment.

Optionally, step S11 can include the following steps.

Step S11a, a gas pressure value at a target position is detected according to the preset sampling period, and a target pressure difference at the target position is determined according to the gas pressure value and an atmospheric pressure value, where the target position is a gas delivery position where the oxygen generation system 10 delivers oxygen to the user.

In the embodiment of the present disclosure, the air supply pipe 104 in the oxygen generation system 10 is used to connect the user and deliver oxygen to the user. The target position can be a pipe outlet position of the air supply pipe 104. The target position can be an outlet of the air supply pipe 104 on a side close to the user, or can also be an outlet of the air supply pipe 104 on a side close to the oxygen generation system 10, which is not limited by the embodiment of the present disclosure.

In the embodiment of the present disclosure, when the user inhales, a pressure difference will be generated in the air supply pipe 104 of the oxygen generation system 10. The oxygen generation system 10 can detect the gas pressure value at the target position according to the preset sampling period, that is, at the certain time interval, and then compare the obtained gas pressure value with the atmospheric pressure value corresponding to a current environment of the oxygen generation system 10 to determine the pressure difference at the target position. The gas pressure value can be subtracted from the atmospheric pressure value to obtain the target pressure difference.

Step S11b, a first difference value is determined according to the target pressure difference and a basic pressure difference, and it is determined that one-time inspiratory action of the user is sampled in response to an absolute value of the first difference value being greater than a preset pressure difference threshold, where the basic pressure difference is a pressure difference calibration value at the target position in a preset time interval after the oxygen generation system 10 supplies oxygen to the user.

In the embodiment of the present disclosure, a preset pressure difference threshold represents a threshold value corresponding to the inspiratory action of the user. The basic pressure difference can be a stable pressure difference at a non-oxygen supply moment in the air supply pipe 104 of the oxygen generation system 10. The oxygen generation system 10 can perform real-time calibration on the basic pressure difference. Specifically, the oxygen generation system 10 can continuously detect the pressure difference in the air supply pipe 104 after supplying oxygen to the user every time, and then perform calibration on the basic pressure difference according to the stable pressure difference in the preset time interval after the pressure difference in the air supply pipe 104 is stabilized. A pressure difference calibration value can be obtained by calculating an average or median value of stable pressure differences in the preset time interval as the basic pressure difference. The preset time interval can be determined according to actual application requirements, for example, an average value of the stable pressure differences in the air supply pipe 104 of the oxygen generation system 10 within ten seconds(s) can be taken as the basic pressure difference.

In the embodiment of the present disclosure, after obtaining the target pressure difference at the target position every time, the oxygen generation system 10 can compare the target pressure difference with the basic pressure difference at the target position to determine a variation of the pressure difference at the target position, that is, the first difference value. The target pressure difference can be subtracted from the basic pressure difference to obtain the first difference value, which can be a negative value. The preset pressure difference threshold can be a positive value, and an actual value can be determined according to application requirements, which is not limited by the embodiments of the present disclosure. The oxygen generation system 10 can compare the absolute value of the first difference value with the preset pressure difference threshold, if the absolute value of the first difference value is greater than the preset pressure difference threshold, one-time valid data is recorded, that is, it is determined that one-time inspiratory action of the user is sampled; and if the absolute value of the first difference value is not greater than the preset pressure difference threshold, the target pressure difference sampled this time is invalid data.

For example, if the first difference value obtained by subtracting the target pressure difference from the basic pressure difference is −30 Pascal (Pa), and the preset pressure difference threshold is set to 20 Pascal (Pa), the absolute value of the first difference value is greater than the preset pressure difference threshold, and it can be determined that one-time inspiratory action of the user is sampled.

In the embodiment of the present disclosure, the gas pressure value at the target position is detected according to the preset sampling period, and the target pressure difference at the target position is determined according to the gas pressure value and the atmospheric pressure value. Since the target position is the gas delivery position where the oxygen generation system 10 delivers oxygen to the user, the oxygen generation system 10 can detect the pressure difference between the gas pressure value at the gas delivery position and the atmospheric pressure value, determine the first difference value according to the target pressure difference and the basic pressure difference, and when the absolute value of the first difference value is greater than the preset pressure difference threshold, it is determined that one-time inspiratory action of the user is sampled, and thus the variation of the pressure difference (that is, the first difference value) at the target position and a relationship between the first difference value and the preset pressure difference threshold can be determined, and whether one-time inspiratory action of the user is sampled can be determined. Since the basic pressure difference is the pressure difference calibration value at the target position in the preset time interval after the oxygen generation system 10 supplies oxygen to the user, the determined first difference value can be more accurate, thereby improving the sampling accuracy of the inspiratory action of the user.

S12, in response to a quantity of times of sampled inspiratory actions being greater than a preset quantity-of-times threshold, it is determined that the inspiratory event of the user is detected, and the detection signal corresponding to the inspiratory event is generated, where the preset quantity-of-times threshold is determined according to a breathing frequency of the user.

In the embodiment of the present disclosure, the preset quantity-of-times threshold can be dynamically set according to the breathing frequency of the user, so that the detection process of the oxygen generation system 10 for the inspiratory event of the user is more adaptive to the user, thereby making the detection signal corresponding to the inspiratory event more accurate. Specifically, for users with a relatively slow breathing frequency, an actual value of the preset quantity-of-times threshold can be increased; and for users with a relatively low breathing frequency, the preset quantity-of-times threshold can be reduced, which can enable the users with a relatively high breathing frequency to trigger faster and the users with the relatively low breathing frequency to make more stable decisions. For example, if the breathing frequency is less than or equal to twenty times per minute, the preset quantity-of-times threshold is set to five times; and if the breathing frequency is greater than twenty times per minute, the preset quantity-of-times threshold is set to three times. This is only an example, and the embodiments of the present disclosure do not limit this.

In the embodiment of the present disclosure, the determination of inspiratory events can be determined by whether the quantity of times of inspiratory actions of the user exceeds a preset quantity of times of determinations, that is, if the quantity of times of sampled inspiratory actions of the user is greater than the preset quantity-of-times threshold, it is determined that one-time inspiratory event of the user is detected. The detection signal corresponding to the inspiratory event is used to represent that the inspiratory event of the user is detected, and the detection time can include a detection time of the inspiratory event. Specifically, the detection time can be a sampling time of the latest inspiratory action of the user.

In the embodiment of the present disclosure, by sampling the inspiratory action of the user according to the preset sampling period, and determining that the inspiratory event of the user is detected in response to the quantity of times of sampled inspiratory actions being greater than the preset quantity-of-times threshold, when the inspiratory actions of the user are accumulated to exceed the preset quantity-of-times threshold, it is determined that one-time inspiratory event of the user is detected, and the detection signal corresponding to the inspiratory event is generated. Since the preset quantity-of-times threshold is determined according to the breathing frequency of the user, the preset quantity-of-times threshold is more suitable for the user, so that the inspiratory event of the user determined according to the preset quantity-of-times threshold is more accurate.

Step S2, an interval duration between a detection time corresponding to a latest detection signal and a current time is determined, and a first control signal is generated in response to the interval duration being greater than a first duration threshold.

In the embodiment of the present disclosure, the latest detection signal can be a detection signal corresponding to the latest inspiratory event of the user. The detection time corresponding to the detection signal can be a generation time of the detection signal, or can be a detection time of the latest inspiratory action of the user in the inspiratory event corresponding to the detection signal. This is only an example, and the embodiments of the present disclosure do not limit this.

In the embodiments of the present disclosure, by determining the interval duration between the detection time corresponding to the latest detection signal and the current time, the interval duration between the latest inspiratory event of the user and the current time can be determined. If the interval duration is greater than a preset first duration threshold, it indicates that the oxygen generation system 10 has not detected the inspiratory event of the user over the first duration threshold, and thus in order to reduce the power consumption of the oxygen generation system 10, the oxygen generation amount can be controlled to be reduced by the first control signal, so as to avoid the excessive power consumption of the oxygen generation system 10 caused by continuously generating oxygen according to an original oxygen generation amount when the inspiratory event of the user is not detected for a certain time. The first control signal can include a target oxygen generation amount.

Step S3, a latest oxygen generation amount of the oxygen generation system 10 is reduced from a first oxygen generation amount to a target oxygen generation amount according to the first control signal, where the first oxygen generation amount is an oxygen generation amount corresponding to a first gear currently set for the oxygen generation system 10.

In the embodiment of the present disclosure, the oxygen generation system 10 can include a plurality of gears, and different gears indicate that oxygen supply amounts of the oxygen generation system 10 to the user are different. Correspondingly, oxygen generation amounts corresponding to different gears are also different. For example, the oxygen generation system 10 can include a first gear to a fifth gear. The higher the gear, the higher the oxygen supply amount, and the higher the oxygen generation amount. The first gear currently set for the oxygen generation system 10 indicates an original set gear set for the oxygen generation system 10 before performing the operation of reducing the latest oxygen generation amount. The first gear can be the lowest gear, the highest gear, or other gears, and this is not limited by the embodiments of the present disclosure.

In the embodiment of the present disclosure, the target oxygen generation amount can be an oxygen generation amount corresponding to a set gear of the oxygen generation system 10 that is lower than the first gear, or the target oxygen generation amount can be zero, which indicates that the oxygen generation system 10 stops oxygen generation and enters a standby state. After the oxygen generation system 10 enters the standby state, hardware related to the oxygen generation process can stop working, and only a unit that detects the inspiratory event of the user can be retained.

In the embodiment of the present disclosure, the oxygen generation system 10 can determine the target oxygen generation amount as the latest oxygen generation amount of the oxygen generation system 10 according to the first control signal, so that the latest oxygen generation amount is reduced from the first oxygen generation amount corresponding to the first gear to the target oxygen generation amount. In this way, since oxygen required to be generated by the oxygen generation system 10 is reduced, the power consumption generated by the oxygen generation system 10 is also reduced.

Optionally, step S2 can include the following steps:

step S21, in response to the interval duration being greater than the first duration threshold and less than a second duration threshold, the oxygen generation system 10 is lowered from the first gear to a second gear, and a first sub-signal corresponding to the second gear is generated.

Step S3 can include the following steps:

step S31, the latest oxygen generation amount is reduced from the first oxygen generation amount to a second oxygen generation amount corresponding to the second gear according to the first sub-signal.

In the embodiment of the present disclosure, the second duration threshold is a set value greater than the first duration threshold. For example, the first duration threshold is one minute (min), and the second duration threshold can be five minutes. When the interval duration is greater than the first duration threshold and less than the second duration threshold, it indicates that the oxygen generation system 10 has not detected the inspiratory event of the user temporarily. In order to reduce the power consumption of the oxygen generation system 10, the set gear of the oxygen generation system 10 can be lowered from the first gear to the second gear. The second gear can be the lowest gear of the oxygen generation system 10, or the second gear can be a set gear lower than the first gear, which is not limited by the embodiments of the present disclosure.

In the embodiment of the present disclosure, it is determined whether the first gear is the lowest gear, and if the first gear is not the lowest gear, the system can lower the gear from the first gear to the second gear, where the second gear is lower than the first gear. The gear adjustment from the first gear to the second gear is not limited to continuous hierarchical adjustment or discontinuous nonhierarchical adjustment until the gear is finally lowered to the lowest gear. For example, when the first gear is in fifth gear, the continuous hierarchical adjustment can be performed according to a predetermined time, that is, the gear is lowered from the fifth gear to the fourth gear, the third gear, the second gear and the first gear; or the discontinuous nonhierarchical adjustment can also be performed according the predetermined time, that is, the gear is lowered from the fifth gear to the third gear and from the third gear to the first gear. It should be noted that all the above methods are within the protection scope of the present disclosure. In addition, if the first gear is the lowest gear, subsequent steps S22 and S32 can be executed.

In the embodiment of the present disclosure, the first sub-signal corresponding to the second gear can include the second oxygen generation amount corresponding to the second gear. The oxygen generation system 10 can determine the latest oxygen generation amount of the oxygen generation system 10 as the second oxygen generation amount according to the first sub-signal, thereby reducing the latest oxygen generation amount from the first oxygen generation amount to the second oxygen generation amount corresponding to the second gear.

In the embodiment of the present disclosure, by lowering the oxygen generation system 10 from the first gear to the second gear in the case where the interval duration is greater than the first duration threshold and less than the second duration threshold, generating the first sub-signal corresponding to the second gear, and reducing the latest oxygen generation amount from the first oxygen generation amount to the second oxygen generation amount corresponding to the second gear according to the first sub-signal, the latest oxygen generation amount can be reduced by lowering the set gear of the oxygen generation system 10, thereby reducing the power consumption of the oxygen generation system 10. Although the oxygen generation amount is reduced, the oxygen generation system 10 does not stop an oxygen generation function, so that when the inspiratory event of the user is detected again, the oxygen generation system 10 can quickly respond to restore oxygen supply for the user.

Optionally, step S2 can include the following steps:

step S22, a second sub-signal is generated in response to the interval duration being greater than the second duration threshold.

Step S3 can include the following steps:

step S32, the latest oxygen generation amount is reduced from the second oxygen generation amount to zero according to the second sub-signal.

In the embodiment of the present disclosure, in the case where the interval duration is greater than the second duration threshold, which indicates that the oxygen generation system 10 has not detected the inspiratory event of the user for a long time, in order to further reduce the power consumption of the oxygen generation system 10, the oxygen generation function can be directly turned off, that is, the latest oxygen generation amount is reduced to zero, so that the oxygen generation system 10 enters the standby state.

In the embodiment of the present disclosure, the second sub-signal can include information that the latest oxygen generation amount is zero. The oxygen generation system 10 can determine the latest oxygen generation amount to be zero according to the second sub-signal, thereby stopping the oxygen generation function. After the oxygen generation system 10 enters the standby state, the hardware related to the oxygen generation process can stop working, and only the unit that detects the inspiratory event of the user can be retained. In this way, the power consumption can be minimized while retaining the function of detecting the inspiratory event of the user, and the oxygen generation system 10 can restore the oxygen generation function and deliver oxygen for the user after the inspiratory event of the user is detected again.

In the embodiment of the present disclosure, by generating the second sub-signal in the case where the interval duration is greater than the second duration threshold, and reducing the latest oxygen generation amount from the second oxygen generation amount to zero according to the second sub-signal, the oxygen generation function of the oxygen generation system 10 can be stopped to avoid the power consumption caused by the oxygen generation process without detecting the inspiratory event of the user for a long time.

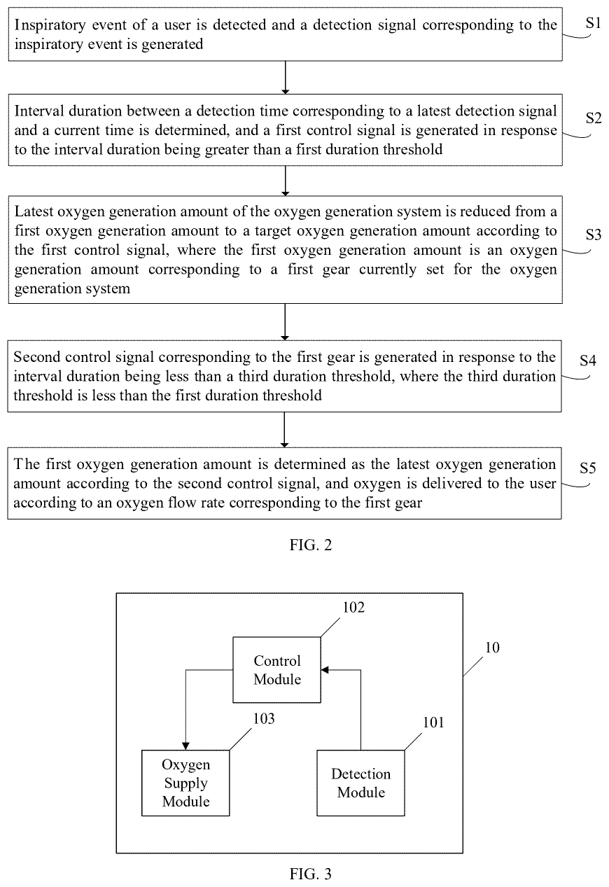

Optionally, referring to FIG. 2, the method for controlling oxygen generation can further include the following steps.

Step S4, a second control signal corresponding to the first gear is generated in response to the interval duration being less than a third duration threshold, where the third duration threshold is less than the first duration threshold.

In the embodiment of the present disclosure, the third duration threshold represents an effective interval duration of the inspiratory event of the user. If the interval duration between the detection time corresponding to the latest detection signal and the current time is less than the third duration threshold, it indicates that the oxygen generation system 10 has detected an effective inspiratory event of the user, and it is necessary for the oxygen generation system 10 to generate oxygen and supply oxygen for the user according to the current set gear (that is, the first gear). The second control signal can include the first oxygen generation amount corresponding to the first gear and an oxygen flow rate of the first gear for supplying oxygen to the user. After the oxygen generation system 10 is lowered from the first gear to the second gear, in the case where the oxygen generation system 10 detects the inspiratory event of the user again and generates a corresponding detection signal, it is determined a relationship between the detection time of the detection signal and the third duration threshold. If the detection time of the detection signal is less than the third duration threshold, it indicates that the oxygen generation system 10 has detected the inspiratory event of the user again, the oxygen generation system 10 can be controlled to return to the original set gear, that is, the first gear, and then oxygen can be generated and supplied to the user according to the first gear.

The third duration threshold is set to be less than the first duration threshold. In practical applications, the third duration threshold can be determined according to the preset sampling period of the oxygen generation system 10 for the inspiratory action of the user. For example, the oxygen generation system 10 collects the pressure difference of the air supply pipe 104 every two milliseconds, and the preset quantity-of-times threshold is five times, and thus the third duration threshold can be set to 30 milliseconds. This is only an example, and the embodiments of the present disclosure do not limit this.

Step S5, the first oxygen generation amount is determined as the latest oxygen generation amount according to the second control signal, and oxygen is delivered to the user according to an oxygen flow rate corresponding to the first gear.

In the embodiment of the present disclosure, the oxygen generation system 10 can generate oxygen based on the principle of pressure swing adsorption, which utilizes selective adsorption characteristics of zeolite molecular sieve and adopts a cycle of pressurized adsorption and depressurized desorption to enable compressed air to alternately enter the adsorption tower for air separation, thereby continuously generating a certain amount of oxygen. The second control signal can include a third sub-signal and a fourth sub-signal. The oxygen generation system 10 can determine an operating pressure of the oxygen generation system 10 according to the first oxygen generation amount indicated by the third sub-signal to generate oxygen with the first oxygen generation amount, and then deliver oxygen to the user through the air supply pipe 104 of the oxygen generation system 10 according to the oxygen flow rate corresponding to the first gear indicated by the fourth sub-signal.

Optionally, step S5 can include the following steps:
step S51, a latest oxygen generation pressure of the oxygen generation system 10 is determined as a target pressure value according to the second control signal, where the target pressure value is an oxygen generation pressure value corresponding to the first oxygen generation amount; and
step S52, oxygen with the first oxygen generation amount is generated according to the latest oxygen generation pressure.

In the embodiment of the present disclosure, the oxygen generation system 10 can determine an operating pressure value (that is, a latest oxygen generation pressure) of the oxygen generation system 10 according to the first oxygen generation amount. The oxygen generation system 10 can include a compressor, and a system pressure can be changed by controlling a rotating speed of the compressor, so that the latest oxygen generation pressure is equal to the target pressure value, and then oxygen with the first oxygen generation amount can be generated based on the principle of pressure swing adsorption.

In the embodiment of the present disclosure, by determining the latest oxygen generation pressure of the oxygen generation system 10 as the target pressure value according to the second control signal, where the target pressure value is the oxygen generation pressure value corresponding to the first oxygen generation amount, and generating oxygen with the first oxygen generation amount according to the latest oxygen generation pressure, the oxygen generation system 10 can change the oxygen generation pressure value of the oxygen generation system 10 based on the second control signal, that is, the latest oxygen generation pressure is determined as the target pressure value, so that the oxygen generation system 10 can generate oxygen with the first oxygen generation amount according to the latest oxygen generation pressure, thereby improving the practicability of the method for controlling oxygen generation in the embodiment of the present disclosure.

In the embodiment of the present disclosure, the second control signal corresponding to the first gear is generated in the case where the interval duration is less than the third duration threshold, since the third duration threshold is less than the first duration threshold, it can be determined that the oxygen generation system 10 has detected the effective inspiratory event of the user, the first oxygen generation amount is determined as the latest oxygen generation amount according to the second control signal, and oxygen is delivered to the user according to the oxygen flow rate corresponding to the first gear. In this way, the oxygen generation system 10 can generate oxygen for the user according to the first gear currently set and based on the second control signal, and deliver oxygen to the user according to the oxygen flow rate corresponding to the first gear, thereby improving the practicability of the method for controlling oxygen generation in the embodiment of the present disclosure.

In the embodiments of the present disclosure, by detecting the inspiratory event of the user, generating the detection signal corresponding to the inspiratory event, and determining the interval duration between the detection time corresponding to the latest detection signal and the current time, the interval duration between the current time and the latest inspiratory event of the user can be determined, and the first control signal is generated in the case where the interval duration is greater than the first duration threshold; then reducing the latest oxygen generation amount of the oxygen generation system 10 from the first oxygen generation amount to the target oxygen generation amount according to the first control signal, the latest oxygen generation amount of the oxygen generation system 10 can be reduced based on the first control signal in the case where the interval duration exceeds the first duration threshold and the inspiratory event of the user is not detected. Since the first oxygen generation amount is the oxygen generation amount corresponding to the first gear currently set for the oxygen generation system 10, by reducing the latest oxygen generation amount from the first oxygen generation amount to a target oxygen generation amount, the problem of excessive power consumption of the oxygen generation system 10 caused by continuously generating oxygen according to the current set gear in the case where the inspiratory event of the user is not detected by the oxygen generation system 10 for a period of time in the related art can be avoid. According to the method for controlling oxygen generation provided in the embodiment of the present disclosure, the power consumption of the oxygen generation system 10 can be reduced by reducing the latest oxygen generation amount of the oxygen generation system 10.

FIG. 3 is a schematic structural diagram of an oxygen generation system according to an exemplary embodiment. As shown in FIG. 3, the oxygen generation system 10 includes a detection module 101, a control module 102 and an oxygen supply module 103, where the control module 102 is connected to the detection module 101 and the oxygen supply module 103 respectively,
the detection module 101 is configured to detect an inspiratory event of a user, generate a detection signal corresponding to the inspiratory event, and transmit the detection signal to the control module 102;
the control module 102 is configured to determine an interval duration between a detection time corresponding to a latest detection signal and a current time, generate a first control signal in response to the interval duration being greater than a first duration threshold, and transmit the first control signal to the oxygen supply module 103; and
the oxygen supply module is configured to reduce a latest oxygen generation amount of the oxygen generation system 10 from a first oxygen generation amount to a target oxygen generation amount according to the first control signal, where the first oxygen generation amount is an oxygen generation amount corresponding to a first gear currently set for the oxygen generation system 10.

In the embodiment of the present disclosure, the control module 102 can establish communication connections with the detection module 101 and the oxygen supply module 103, respectively. The communication connections can be wired connections or wireless connections, and the embodiments of the present disclosure do not limited this. The detection module 101 can detect the inspiratory event of the user and generate the detection signal corresponding to the inspiratory event. The detection method of the inspiratory event can refer to related descriptions of step S1 in the embodiments of the method for controlling oxygen generation mentioned above, which will not be repeated here. The detection module can transmit the detection signal of inspiratory event to the control module 102 through the communication connection established with the control module 102. Specifically, after one-time inspiratory event is detected, the detection module 101 transmits the detection signal of this inspiratory event to the control module 102 in real time. A signal transmission delay of the detection module 101 should be less than a delay threshold, so that the control module 102 can more accurately determine the interval duration between the latest inspiratory event of the user and the current time according to the latest detection signal after receiving the detection signal.

Optionally, the detection module 101 is specifically configured to:
sample an inspiratory action of the user according to a preset sampling period; and
in response to a quantity of times of sampled inspiratory actions being greater than a preset quantity-of-times threshold, determine that the inspiratory event of the user is detected, and generate the detection signal corresponding to the inspiratory event, where the preset quantity-of-times threshold is determined according to a breathing frequency of the user.

In the embodiment of the present disclosure, the oxygen generation system 10 can detect changes of pressure or flow rate in the air supply pipe 104 through the detection module 101, so as to detect the inspiratory event of the user. The specific detection method of the changes of pressure or flow rate can refer to the related descriptions in step S1 in the previous embodiments, which will not be repeated here.

Optionally, the oxygen generation system 10 further includes the air supply pipe 104, where a first end of the air supply pipe 104 is connected to the oxygen supply module 103 and the detection module 101 respectively, a second end of the air supply pipe 104 is connected to the user, and the detection module 101 is further specifically configured to:
detect a gas pressure value in the air supply pipe 104 according to the preset sampling period, and determine a target pressure difference in the air supply pipe 104 according to the gas pressure value and an atmospheric pressure value; and
determine a first difference value according to the target pressure difference and a basic pressure difference, and determine the inspiratory action of the user is sampled in response to an absolute value of the first difference value being greater than a preset pressure difference threshold, where the basic pressure difference is a pressure difference calibration value of the air supply pipe 104 in a preset time interval after the oxygen generation system 10 supplies oxygen to the user.

In the embodiment of the present disclosure, the oxygen generation system 10 further includes the air supply pipe 104, where the first end of the air supply pipe 104 is connected to the oxygen supply module 103, the second end of the air supply pipe 104 is configured to connect the user, and the oxygen supply module 103 can deliver oxygen to the user through the air supply pipe 104. The first end of the air supply pipe 104 is connected to an oxygen output unit 1033. The detection module 101 can include a pressure difference sensor. One end of the pressure difference sensor can be arranged in the air supply pipe 104 for testing the pressure in the air supply pipe 104, and the other end of the pressure difference sensor is communicated with the atmosphere for testing the pressure difference between the pressure in the air supply pipe 104 and the atmospheric pressure. The detection module 101 can further include a control unit, which is configured to perform data processing on a pressure difference detected by the pressure difference sensor according to inspiratory detection algorithms, generate a detection signal corresponding to the inspiratory event after determining that the inspiratory event of the user is detected, and transmit the detection signal to the control module 102. The inspiratory detection algorithms can refer to related descriptions of steps S11 to S12 in the embodiments of the method for controlling oxygen generation mentioned above, which will not be repeated here.

In the embodiment of the present disclosure, the control module 102 is configured to receive the detection signal of inspiratory event transmitted by the detection module 101, and determine whether the user is using the oxygen generation system 10 according to the detection signal. If the user is using the oxygen generation system 10, the oxygen generation system 10 will enter a working state; and if the user is no longer using the oxygen generation system 10, the oxygen generation system 10 will enter the standby state. The oxygen generation system 10 can stop the oxygen generation function in the standby state, and only needs to maintain a normal operation of the detection module 101 to detect the inspiratory event of the user, thereby reducing the system power consumption of the oxygen generation system 10.

In the embodiment of the present disclosure, the oxygen generation system 10 can include a plurality of gears, and different gears indicate that oxygen supply amounts of the oxygen generation system 10 to the user are different. Correspondingly, oxygen generation amounts corresponding to different gears are also different. For example, the oxygen generation system 10 can include a first gear to a fifth gear. The higher the gear, the higher the oxygen supply amount, and the higher the oxygen generation amount.

In the embodiment of the present disclosure, the control module 102 can determine the interval duration between the latest inspiratory event of the user and the current time by determining the interval duration between the detection time corresponding to the latest detection signal and the current time. If the interval duration is greater than a preset first duration threshold, it indicates that the oxygen generation system 10 has not detected the inspiratory event of the user over the first duration threshold, and thus in order to reduce the power consumption of the oxygen generation system 10, the oxygen generation amount can be controlled to be reduced by the first control signal, so as to avoid the problem of the excessive power consumption of the oxygen generation system 10 caused by continuously generating oxygen according to an original oxygen generation amount when the inspiratory event of the user is not detected for a certain time. Control logics executed by the control module 102 can refer to related descriptions of step S2 and step S4 in the embodiments of the method for controlling oxygen generation mentioned above, which will not be repeated here.

In the embodiment of the present disclosure, the oxygen generation system 10 further includes the air supply pipe 104, where a first end of the air supply pipe 104 is connected to the oxygen supply module 103 and the detection module 101, respectively; and a second end of the air supply pipe 104 is connected to the user. By using the detection module 101 to detect the gas pressure value in the air supply pipe 104 according to the preset sampling period, and determining the corresponding target pressure difference in the air supply pipe 104 according to the gas pressure value and the atmospheric pressure value, the variation of the pressure difference (that is, the first difference value) at the target position and a relationship between the first difference value and the preset pressure difference threshold can be determined, and thus whether one-time inspiratory action of the user is sampled can be determined. Since the basic pressure difference is the pressure difference calibration value in the air supply pipe 104 within the preset time interval after the oxygen generation system 10 supplies oxygen to the user, the determined first difference value can be more accurate, thereby improving the sampling accuracy of the inspiratory action of the user.

In the embodiment of the present disclosure, by using the detection module 101 to sample the inspiratory action of the user according to the preset sampling period, and determining that the inspiratory event of the user is detected in response to the quantity of times of sampled inspiratory actions being greater than the preset quantity-of-times threshold, when the inspiratory actions of the user are accumulated to exceed the preset quantity-of-times threshold, it is determined that one-time inspiratory event of the user is detected, and the detection signal corresponding to the inspiratory event is generated. Since the preset quantity-of-times threshold is determined according to the breathing frequency of the user, the preset quantity-of-times threshold is more suitable for the user, so that the inspiratory event of the user determined according to the preset quantity-of-times threshold is more accurate.

Optionally, the control module 102 is specifically configured to:

in response to the interval duration being greater than the first duration threshold and less than a second duration threshold, lower the oxygen generation system 10 from the first gear to a second gear, and generate a first sub-signal corresponding to the second gear.

In the embodiment of the present disclosure, after determining the interval duration corresponding to the latest detection signal, the control module 102 can determine a control strategy according to relationships between the interval duration with the first duration threshold, the second duration threshold and the third duration threshold, and generate a corresponding control signal. Specifically, when the interval duration is greater than the first duration threshold and less than the second duration threshold, it indicates that the oxygen generation system 10 has not detected the inspiratory event of the user temporarily, the control module 102 can lower the current set gear of the oxygen generation system 10 from the first gear to the second gear and generate the first sub-signal, and then transmit the first sub-signal to the oxygen supply module 103, so as to control the oxygen supply module 103 to reduce the latest oxygen generation amount from the first oxygen generation amount to the second oxygen generation amount corresponding to the second gear according to the first sub-signal, and generate oxygen with the second oxygen generation amount.

Optionally, the control module 102 is further specifically configured to:

generate a second sub-signal in response to the interval duration being greater than the second duration threshold

In the embodiment of the present disclosure, when the interval duration is greater than the second duration threshold, it indicates that the oxygen generation system 10 has not detected the inspiratory event of the user for a long time, and the control module 102 can control the oxygen generation system 10 to enter the standby state. Specifically, the control module 102 can generate the second sub-signal and transmit the second sub-signal to the oxygen supply module 103 to control the oxygen supply module 103 to reduce the latest oxygen generation amount from the second oxygen generation amount to zero according to the second sub-signal, thereby stopping the oxygen generation function of the oxygen generation system 10 and reducing the system power consumption.

Optionally, the control module 102 is further configured to generate a second control signal corresponding to the first gear in response to the interval duration being less than a third duration threshold, where the third duration threshold is less than the first duration threshold.

In the embodiment of the present disclosure, when the interval duration is less than the third duration threshold, it indicates that the detection module 101 has detected an effective inspiratory event of the user, and the control module 102 can maintain the current set gear of the oxygen generation system 10 and control the oxygen supply module 103 to work according to the first gear currently set. Specifically, the control module 102 can generate the second control signal corresponding to the first gear and transmit the second control signal to the oxygen supply module 103, so as to control the oxygen supply module 103 to determine the first oxygen generation amount as the latest oxygen generation amount according to the second control signal, and deliver oxygen to the user according to the oxygen flow rate corresponding to the first gear.

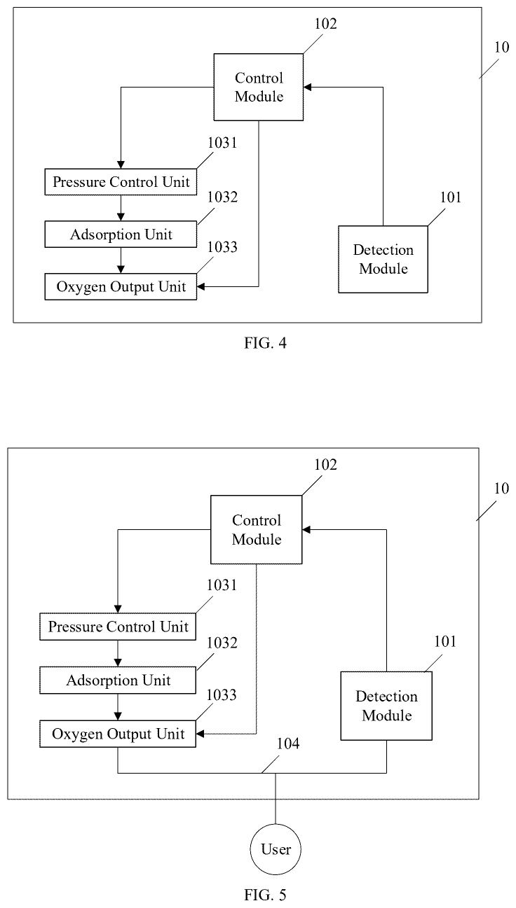

In the embodiment of the present disclosure, referring to FIG. 4, the oxygen supply module 103 can include a pressure control unit 1031, an adsorption unit 1032 and the oxygen output unit 1033. The pressure control unit 1031 is configured to control a latest oxygen generation pressure during operation, the adsorption unit 1032 is configured to generate a corresponding amount of oxygen according to the latest oxygen generation pressure and based on the principle of pressure swing adsorption, and the oxygen output unit 1033 can be connected to the air supply pipe for supplying oxygen to the user according to the current set gear of the oxygen generation system 10. The principle of pressure swing adsorption can refer to related descriptions of step S5 in the embodiments of the method for controlling oxygen generation mentioned above, which will not be repeated here.

In the embodiment of the present disclosure, the first control signal transmitted by the control module 102 can be received by the pressure control unit 1031, and an actual value of the latest oxygen generation pressure can be adjusted by the pressure control unit 1031 in response to the first control signal, thereby changing an actual amount of oxygen generated by the adsorption unit 1032, so that the latest oxygen generation amount of the oxygen generation system 10 is reduced from the first oxygen generation amount corresponding to the first gear currently set to the target oxygen generation amount. The detailed description of the oxygen supply module 103 can refer to related descriptions of step S3 in the embodiments of the method for controlling oxygen generation mentioned above, which will not be repeated here.

Optionally, the oxygen supply module 103 is specifically configured to reduce the latest oxygen generation amount from the first oxygen generation amount to a second oxygen generation amount corresponding to the second gear according to the first sub-signal.

Optionally, the oxygen supply module 103 is further specifically configured to reduce the latest oxygen generation amount from the second oxygen generation amount to zero according to the second sub-signal.

Optionally, the oxygen supply module 103 is further configured to determine the first oxygen generation amount as the latest oxygen generation amount according to the second control signal, and deliver oxygen to the user according to an oxygen flow rate corresponding to the first gear.

In the embodiment of the present disclosure, the first sub-signal transmitted by the control module 102 can be received by the pressure control unit 1031, and the pressure control unit 1031 can adjust an output latest oxygen generation pressure to an oxygen generation pressure value corresponding to the second oxygen generation amount, so that the adsorption unit 1032 can generate oxygen with the second oxygen generation amount according to the oxygen generation pressure value corresponding to the second oxygen generation amount, thereby reducing the latest oxygen generation amount of the oxygen generation system 10 from the first oxygen generation amount to the second oxygen generation amount corresponding to the second gear. The detailed description can refer to related descriptions of step S31 in the embodiments of the method for controlling oxygen generation mentioned above, which will not be repeated here.

In the embodiment of the present disclosure, the second sub-signal transmitted by the control module 102 can be received by the pressure control unit 1031, and in response to the second sub-signal, the pressure control unit 1031 can adjust the output latest oxygen generation pressure to zero, that is, the pressure control unit 1031 stops working, and the adsorption unit 1032 also stops working, so that the latest oxygen generation amount of the oxygen generation system 10 is reduced to zero, and the oxygen generation function of the oxygen generation system 10 is turned off. The detailed description can refer to related descriptions of step S32 in the embodiments of the method for controlling oxygen generation mentioned above, which will not be repeated here.

Referring to Table 1, the oxygen generation system 10 can include a working state and a standby state. In the working state, when the detection module 101 detects the inspiratory event of the user, the control module 102 can transmit a control signal to the oxygen supply module 103 according to the set gear, so that the oxygen supply module 103 generates oxygen and supplies oxygen to the user, and the system power consumption is normal.

TABLE 1
Control logic of oxygen generation system

| System state | Inspiratory event | System control | System |
|---|---|---|---|
| Working state | Yes | Maintain the set gear | Power consumption |
| | | | is normal |
| | No | Duration t1, the system is | Power consumption |
| | | reduced to the first gear | is initially reduced |
| | | Duration t2, the system enters | Power consumption |
| | | the standby state | is minimized |
| Standby state | Yes | Restore the set gear, and | Power consumption |
| | | transmit the control signal | is normal |
| | No | Maintain the shutdown | Power consumption |
| | | control signal, and the system | is maintained to a |
| | | maintains the standby state | minimum |

Referring to Table 1, when the detection module 101 does not detect the inspiratory event of the user for a duration t1, the control module 102 can lower the set gear of the oxygen generation system 10 to the lowest gear, that is, the first gear, and transmit the control signal corresponding to the first gear to the oxygen supply module 103, so that the oxygen supply module 103 can reduce the oxygen generation amount, thereby initially reducing the system power consumption. When the detection module 101 does not detect the inspiratory event of the user for a duration t2, the control module 102 can control the oxygen supply module 103 to stop generating oxygen, thereby controlling the oxygen generation system to enter the standby state, and minimizing the power consumption.

In Table 1, when the oxygen generation system 10 is in standby state, the hardware related to the oxygen generation process in the oxygen generation system 10 stops working, and only the unit that detects the inspiratory event of the user continues to work. When the oxygen generation system 10 detects the inspiratory event of the user again in standby state and generates a corresponding detection signal, the detection time of the detection signal is less than the third duration threshold, which indicates that the oxygen generation system 10 has detected the inspiratory event of the user again, the oxygen generation system 10 can start the oxygen generation function, restore the oxygen generation system 10 to the set gear such as the first gear, and transmit a control signal such as the second control signal corresponding to the first gear to make the oxygen generation system 10 resume oxygen generation and deliver oxygen to the user. At this time, the power consumption of the oxygen production system 10 is normal power consumption in working state. Specifically, the second control signal can include a third sub-signal and a fourth sub-signal. The second control signal can include a third sub-signal and a fourth sub-signal. The oxygen generation system 10 can determine an operating pressure of the oxygen generation system 10 according to the first oxygen generation amount indicated by the third sub-signal to generate oxygen with the first oxygen generation amount, and the oxygen production system 10 can deliver oxygen to the user through the air supply pipe 104 of the oxygen generation system 10 according to the oxygen flow rate corresponding to the first gear indicated by the fourth sub-signal.

In Table 1, in the case where the oxygen generation system 10 does not detect the inspiratory event of the user for a long time, the oxygen generation system 10 maintains generation and transmission functions of a shutdown control signal in the standby state, for example, stopping the transmission of the second control signal, so that the oxygen generation system 10 maintains the power consumption at the lowest value in the standby state. In this way, it is possible to avoid the problem of excessive power consumption caused by retaining the oxygen generation function in the case where the inspiratory event of the user is not detected by the oxygen generation system 10 for a long time. By controlling the control logic of the oxygen generation system 10 to lower the set gear or enter the standby state, the power consumption of the oxygen generation system 10 can be reasonably reduced.

In the embodiment of the present disclosure, by using the control module 102 to lower the oxygen generation system 10 from the first gear to the second gear in the case where the interval duration is greater than the first duration threshold and less than the second duration threshold, and generating the first sub-signal corresponding to the second gear, and by using the oxygen supply module 103 to reduce the latest oxygen generation amount from the first oxygen generation amount to the second oxygen generation amount corresponding to the second gear according to the first sub-signal, the power consumption of the oxygen generation system 10 can be initially reduced by lowering the set gear of the oxygen generation system 10 and reducing the latest oxygen generation amount. Although the set gear is lowered, the oxygen generation system 10 does not stop the oxygen generation function, and the oxygen supply module 103 can still generate oxygen with the second oxygen generation amount, so that the oxygen generation system 10 can quickly respond to restore oxygen supply for the user when the inspiratory event of the user is detected again.

In the embodiment of the present disclosure, by using the control module 102 to generate the second sub-signal in the case where the interval duration is greater than the second duration threshold, and reducing the latest oxygen generation amount from the second oxygen generation amount to zero according to the second sub-signal, the oxygen supply module 103 can be enabled to stop working, thereby avoiding unnecessary system power consumption caused by the oxygen generation process of the oxygen supply module 103 in the case where the inspiratory event of the user is not detected for a long time.

In the embodiment of the present disclosure, the control module 102 generates the second control signal corresponding to the first gear in the case where the interval duration is less than the third duration threshold, since the third duration threshold is less than the first duration threshold, it can be determined that the oxygen generation system 10 has detected the effective inspiratory event of the user, and the oxygen supply module 103 determines the first oxygen generation amount as the latest oxygen generation amount according to the second control signal, and oxygen is delivered to the user according to the oxygen flow rate corresponding to the first gear. In this way, the oxygen generation system 10 can generate oxygen for the user according to the first gear currently set, and deliver oxygen to the user according to the oxygen flow rate corresponding to the first gear, thereby improving the practicability of the method for controlling oxygen generation in the embodiment of the present disclosure.

Optionally, the oxygen supply module 103 includes the pressure control unit 1031 and the adsorption unit 1032, where the control module 102 is connected to the pressure control unit 1031, and the pressure control unit 1031 is further connected to the adsorption unit 1032;
the pressure control unit 1031 is configured to determine a latest oxygen generation pressure of the oxygen generation system 10 as a target pressure value according to the second control signal, where the target pressure value is an oxygen generation pressure value corresponding to the first oxygen generation amount; and
the adsorption unit 1032 is configured to generate oxygen with the first oxygen generation amount according to the latest oxygen generation pressure.

In the embodiment of the present disclosure, the pressure control unit 1031 can include a compressor, and the adsorption unit 1032 has an adsorbent inside. The pressure control unit 1031 receives the control signal transmitted by the control module 102, and can change a speed of the compressor in response to the control signal, thereby changing the latest oxygen generation pressure of the oxygen generation system 10, so that the adsorption unit 1032 can generate oxygen with the second oxygen generation amount corresponding to the second gear according to the reduced latest oxygen generation pressure.

Specifically, the pressure control unit 1031 receives the first sub-signal transmitted by the control module 102, and can reduce the speed of the compressor in response to the first sub-signal, thereby reducing the latest oxygen generation pressure of the oxygen generation system 10 to the oxygen generation pressure value corresponding to the second oxygen generation amount. The pressure control unit 1031 receives the second sub-signal transmitted by the control module 102, and can control the compressor to stop working in response to the second sub-signal, thereby turning off the oxygen generation function of the oxygen generation system 10.

In the embodiment of the present disclosure, the pressure control unit 1031 receives the third sub-signal transmitted by the control module 102, and can change the speed of the compressor in response to the third sub-signal, so that the latest oxygen generation pressure of the oxygen generation system 10 matches the oxygen generation pressure value corresponding to the first oxygen generation amount, that is, the target pressure value, and then the adsorption unit 1032 generates the oxygen with the first oxygen generation amount corresponding to the first gear according to the reduced latest oxygen generation pressure. The detailed description can refer to the related descriptions of step S5 in the embodiments of the method for controlling oxygen generation mentioned above, which will not be repeated here.

In the embodiment of the present disclosure, the oxygen supply module 103 further includes the oxygen output unit 1033, which is connected to the adsorption unit 1032. The oxygen output unit 1033 is configured to deliver oxygen to the user according to the oxygen flow rate corresponding to the first gear and based on the second control signal. The oxygen output unit 1033 can include a control unit, a pressure sensor and an oxygen storage tank. The oxygen storage tank is configured to store a certain amount of oxygen; the pressure sensor is connected to the oxygen storage tank for detecting a pressure inside the oxygen storage tank; and the control unit is configured to receive the fourth sub-signal transmitted by the control module 102, and deliver oxygen to the user through the air supply pipe 104 according to the oxygen flow rate corresponding to the set gear indicated by the fourth sub-signal, as shown in FIG. 5, oxygen is delivered to the user through the air supply pipe 104. The air supply pipe 104 can be provided with a solenoid valve. The solenoid valve can be electrically connected to the control unit inside the oxygen output unit 1033, and the control unit can turn on the solenoid valve in response to the fourth sub-signal to supply oxygen for the user when inhaling.

In the embodiment of the present disclosure, the oxygen supply module 103 includes the pressure control unit 1031 and the adsorption unit 1032. The latest oxygen generation pressure of the oxygen generation system 10 can be determined as the target pressure value by the pressure control unit 1031 according to the second control signal. Since the target pressure value is the oxygen generation pressure value corresponding to the first oxygen generation amount, the adsorption unit 1032 can generate oxygen with the first oxygen generation amount according to the latest oxygen generation pressure, thereby improving the practicability of the oxygen generation system 10.

In the embodiment of the present disclosure, the oxygen generation system 10 includes the detection module 101, the control module 102 and the oxygen supply module 103, where the control module 102 is connected to the detection module 101 and the oxygen supply module 103, respectively. By using the detection module 101 to detect the inspiratory event of the user and generate the detection signal corresponding to the inspiratory event, and using the control module 102 to determine the interval duration between the detection time corresponding to the latest detection signal and the current time, the interval duration between the current time and one-time latest inspiratory event of the user can be determined, and the first control signal is generated in the case where the interval duration is greater than the first duration threshold, further, by using the oxygen supply module 103 to reduce the latest oxygen generation amount of the oxygen generation system 10 from the first oxygen generation amount to the target oxygen generation amount according to the first control signal, the latest oxygen generation amount of the oxygen generation system 10 can be reduced based on the first control signal in the case where the interval duration exceeds the first duration threshold and the inspiratory event of the user is not detected. Since the first oxygen generation amount is the oxygen generation amount corresponding to the first gear currently set for the oxygen generation system 10, by reducing the latest oxygen generation amount from the first oxygen generation amount to a target oxygen generation amount, the problem of excessive power consumption of the oxygen generation system 10 caused by continuously generating oxygen according to the current set gear in the case where the inspiratory event of the user is not detected by the oxygen generation system 10 for a period of time in the related art can be avoid. According to the method for controlling oxygen generation provided in the embodiment of the present disclosure, the power consumption of the oxygen generation system 10 can be reduced by reducing the latest oxygen generation amount of the oxygen generation system 10.

Algorithms and displays provided herein are not inherently related to any particular computer, a virtual system, or another device. Various general purpose systems may also be used together with teachings herein. According to the foregoing descriptions, a structure required for constructing such a system is obvious. In addition, the present disclosure is not specifically limited to any particular programming language. It should be understood that the content in the present disclosure described herein may be implemented by using various programming languages, and the foregoing description of the particular language is intended to disclose an optimal implementation of the present disclosure.

A large number of specific details are described in the specification provided herein. However, it can be understood that the embodiments of the present disclosure may be implemented without these specific details. In some examples, known methods, structures, and technologies are not disclosed in detail, so as not to obscure understanding on the specification.

Similarly, it should be understood that to simplify the present disclosure and help to understand one or more of the inventive aspects, in the foregoing descriptions of the exemplary embodiments of the present disclosure, features of the present disclosure are sometimes grouped into a single embodiment or figure, or descriptions thereof. However, the methods in the present disclosure should not be construed as reflecting the following intention: that is, the present disclosure claimed to be protected is required to have more features than those clearly set forth in each claim. Or rather, as reflected in the following claims, the inventive aspects aim to be fewer than all features of a single embodiment disclosed above. Therefore, the claims complying with a specific implementation are definitely combined into the specific implementation, and each claim is used as a single embodiment of the present disclosure.

A person skilled in the art may understand that modules in the device in the embodiments may be adaptively changed and disposed in one or more devices different from that in the embodiments. Modules, units, or components in the embodiments may be combined into one module, unit, or component, and moreover, may be divided into a plurality of sub-modules, subunits, or subcomponents. Unless that at least some of such features and/or processes or units are mutually exclusive, all features disclosed in this specification (including the appended claims, abstract, and drawings) and all processes or units in any disclosed method or device may be combined by using any combination. Unless otherwise definitely stated, each feature disclosed in this specification (including the appended claims, abstract, and drawings) may be replaced with a replacement feature providing a same, an equivalent, or a similar objective.

The component embodiments of the present disclosure may be implemented by using hardware, may be implemented by using software modules running on one or more processors, or may be implemented by using a combination thereof. A person skilled in the art should understand that some or all functions of some or all components of the document server according to the embodiments of the present disclosure may be implemented by using a microprocessor or a digital signal processor (DSP) in practice. The present disclosure may further be implemented as a device or an apparatus program configured to perform some or all of the methods described herein. Such program for implementing the present disclosure may be stored on a computer readable medium, or may have one or more signal forms. Such signals may be downloaded from an Internet website, may be provided from a carrier signal, or may be provided in any other forms.

It should be noted that the foregoing embodiments are descriptions of the present disclosure rather than limitations on the present disclosure, and a person skilled in the art may design a replacement embodiment without departing from the scope of the appended claims. In the claims, any reference symbol located between brackets should not constitute a limitation on the claims. The word “comprise” does not exclude an element or a step not listed in the claims. The word “a” or “one” located before an element does not exclude existence of a plurality of such elements. The present disclosure may be implemented by hardware including several different elements and an appropriately programmed computer. In the unit claims listing several apparatuses, some of the apparatuses may be specifically embodied by using the same hardware. Use of the words such as “first”, “second”, and “third” does not indicate any sequence. These words may be construed as names.

It can be clearly understood by a person skilled in the art that for the purpose of convenient and brief description, for a detailed working process of the foregoing system, apparatus, and unit, refer to a corresponding process in the foregoing method embodiments, and details are not described herein again.

The above is only preferred embodiments of the present disclosure, and is not intended to limit the application. Any modifications, equivalent replacements and improvements made within the spirit and principles of the present disclosure fall within the scope of protection of the present disclosure.

The foregoing descriptions are merely specific implementations of the present disclosure, but are not intended to limit the protection scope of the present disclosure. Any variation or replacement readily figured out by a person skilled in the art within the technical scope disclosed in the present disclosure shall fall within the protection scope of the present disclosure. Therefore, the protection scope of the present disclosure should be subject to the protection scope of the claims.

It should be noted that in the embodiments of the present disclosure, all the processes related to obtaining various data are carried out under the premise of complying with the corresponding data protection laws and policies of the local country and obtaining authorization from the corresponding device owner.

1. A method for controlling oxygen generation, applied to an oxygen generation system, comprising:
detecting an inspiratory event of a user and generating a detection signal corresponding to the inspiratory event;
determining an interval duration between a detection time corresponding to a latest detection signal and a current time, and generating a first control signal in response to the interval duration being greater than a first duration threshold; and
reducing a latest oxygen generation amount of the oxygen generation system from a first oxygen generation amount to a target oxygen generation amount according to the first control signal, wherein the first oxygen generation amount is an oxygen generation amount corresponding to a first gear currently set for the oxygen generation system.

2. The method for controlling oxygen generation according to claim 1, wherein generating the first control signal in response to the interval duration being greater than the first duration threshold comprises:
in response to the interval duration being greater than the first duration threshold and less than a second duration threshold, lowering the oxygen generation system from the first gear to a second gear, and generating a first sub-signal corresponding to the second gear; and
reducing the latest oxygen generation amount of the oxygen generation system from the first oxygen generation amount to the target oxygen generation amount according to the first control signal comprises:
reducing the latest oxygen generation amount from the first oxygen generation amount to a second oxygen generation amount corresponding to the second gear according to the first sub-signal.

3. The method for controlling oxygen generation according to claim 2, wherein generating the first control signal in response to the interval duration being greater than the first duration threshold comprises:
generating a second sub-signal in response to the interval duration being greater than the second duration threshold; and
reducing the latest oxygen generation amount of the oxygen generation system from the first oxygen generation amount to the target oxygen generation amount according to the first control signal comprises:
reducing the latest oxygen generation amount from the second oxygen generation amount to zero according to the second sub-signal.

4. The method for controlling oxygen generation according to claim 1, further comprising:
generating a second control signal corresponding to the first gear in response to the interval duration being less than a third duration threshold, wherein the third duration threshold is less than the first duration threshold; and
determining the first oxygen generation amount as the latest oxygen generation amount according to the second control signal, and delivering oxygen to the user according to an oxygen flow rate corresponding to the first gear.

5. The method for controlling oxygen generation according to claim 4, wherein
determining the first oxygen generation amount as the latest oxygen generation amount according to the second control signal comprises:
determining a latest oxygen generation pressure of the oxygen generation system as a target pressure value according to the second control signal, wherein the target pressure value is an oxygen generation pressure value corresponding to the first oxygen generation amount; and
generating oxygen with the first oxygen generation amount according to the latest oxygen generation pressure.

6. The method for controlling oxygen generation according to claim 1, wherein detecting the inspiratory event of the user and generating the detection signal corresponding to the inspiratory event comprises:
sampling an inspiratory action of the user according to a preset sampling period; and
in response to a quantity of times of sampled inspiratory actions being greater than a preset quantity-of-times threshold, determining that the inspiratory event of the user is detected, and generating the detection signal corresponding to the inspiratory event, wherein the preset quantity-of-times threshold is determined according to a breathing frequency of the user.

7. The method for controlling oxygen generation according to 6, wherein sampling the inspiratory action of the user according to the preset sampling period comprises:
detecting a gas pressure value at a target position according to the preset sampling period, and determining a target pressure difference at the target position according to the gas pressure value and an atmospheric pressure value, wherein the target position is a gas delivery position where the oxygen generation system delivers oxygen to the user; and
determining a first difference value according to the target pressure difference and a basic pressure difference, and determining that one-time inspiratory action of the user is sampled in response to an absolute value of the first difference value being greater than a preset pressure difference threshold, wherein the basic pressure difference is a pressure difference calibration value at the target position in a preset time interval after the oxygen generation system supplies oxygen to the user.

8. An oxygen generation system, comprising: a detector, a controller and an oxygen supplier, wherein the controller is connected to the detector and the oxygen supplier respectively,
the detector is configured to detect an inspiratory event of a user, generate a detection signal corresponding to the inspiratory event, and transmit the detection signal to the controller;
the controller is configured to determine an interval duration between a detection time corresponding to a latest detection signal and a current time, generate a first control signal in response to the interval duration being greater than a first duration threshold, and transmit the first control signal to the oxygen supplier; and
the oxygen supplier is configured to reduce a latest oxygen generation amount of the oxygen generation system from a first oxygen generation amount to a target oxygen generation amount according to the first control signal, wherein the first oxygen generation amount is an oxygen generation amount corresponding to a first gear currently set for the oxygen generation system.

9. The oxygen generation system according to claim 8, wherein the controller is further configured to in response to the interval duration being greater than the first duration threshold and less than a second duration threshold, lower the oxygen generation system from the first gear to a second gear, and generate a first sub-signal corresponding to the second gear; and
the oxygen supplier is further configured to reduce the latest oxygen generation amount from the first oxygen generation amount to a second oxygen generation amount corresponding to the second gear according to the first sub-signal.

10. The oxygen generation system according to claim 9, wherein the controller is further configured to generate a second sub-signal in response to the interval duration being greater than the second duration threshold; and
the oxygen supplier is further configured to reduce the latest oxygen generation amount from the second oxygen generation amount to zero according to the second sub-signal.

11. The oxygen generation system according to claim 8, wherein the controller is further configured to generate a second control signal corresponding to the first gear in response to the interval duration being less than a third duration threshold, wherein the third duration threshold is less than the first duration threshold; and
the oxygen supplier is further configured to determine the first oxygen generation amount as the latest oxygen generation amount according to the second control signal, and deliver oxygen to the user according to an oxygen flow rate corresponding to the first gear.

12. The oxygen generation system according to claim 11, wherein the oxygen supplier comprises a pressure control unit and an adsorption unit, wherein the controller is connected to the pressure control unit, and the pressure control unit is further connected to the adsorption unit,
the pressure control unit is configured to determine a latest oxygen generation pressure of the oxygen generation system as a target pressure value according to the second control signal, wherein the target pressure value is an oxygen generation pressure value corresponding to the first oxygen generation amount; and
the adsorption unit is configured to generate oxygen with the first oxygen generation amount according to the latest oxygen generation pressure.

13. The oxygen generation system according to claim 8, wherein the detector is configured to:
sample an inspiratory action of the user according to a preset sampling period; and
in response to a quantity of times of sampled inspiratory actions being greater than a preset quantity-of-times threshold, determine that the inspiratory event of the user is detected, and generate the detection signal corresponding to the inspiratory event, wherein the preset quantity-of-times threshold is determined according to a breathing frequency of the user.

14. The oxygen generation system according to claim 13, further comprising: an air supply pipe, wherein a first end of the air supply pipe is connected to the oxygen supplier and the detector respectively, a second end of the air supply pipe is connected to the user, and the detector is further configured to:
detect a gas pressure value in the air supply pipe according to the preset sampling period, and determine a target pressure difference in the air supply pipe according to the gas pressure value and an atmospheric pressure value; and
determine a first difference value according to the target pressure difference and a basic pressure difference, and determine one-time inspiratory action of the user is sampled in response to an absolute value of the first difference value being greater than a preset pressure difference threshold, wherein the basic pressure difference is a pressure difference calibration value of the air supply pipe in a preset time interval after the oxygen generation system supplies oxygen to the user.
